SEQUENCE LISTING (17 sequences)

SEQ ID NO: 1 - DNA, 23 bp, ARTIFICIAL SEQUENCE
  SYNTHETIC DNA
caaagaagcttaataatcatatt

SEQ ID NO: 2 - DNA, 19 bp, ARTIFICIAL SEQUENCE
  SYNTHETIC DNA
ttgaccaactggctgagcc

SEQ ID NO: 3 - DNA, 161 bp, ARTIFICIAL SEQUENCE
  SYNTHETIC DNA
gtaccatgtacccatacgacgttccagactacgcttctttgggtggttctagcccaagctcagagctccaccgcggtggcggccgcatcttttacccatacgatgttcctgactatgcgggctatccctatgacgtcccggactatgcaggatccacgtat

SEQ ID NO: 4 - DNA, 28 bp, ARTIFICIAL SEQUENCE
  SYNTHETIC DNA
acgcgaattcatgtctaaaggtgaatta

SEQ ID NO: 5 - DNA, 28 bp, ARTIFICIAL SEQUENCE
  SYNTHETIC DNA
acgcgaattctttgtacaattcatccat

SEQ ID NO: 6 - DNA, 62 bp, ARTIFICIAL SEQUENCE
  SYNTHETIC DNA
tgaaattgttgaatgacattaagagacggaacatgggcaggtctaaaggtgaagaattattc

SEQ ID NO: 7 - DNA, 61 bp, ARTIFICIAL SEQUENCE
  SYNTHETIC DNA
agtctgtggaatataaacggtccttgatcaatgcggagtgttatttgtacaattcatccat

SEQ ID NO: 8 - DNA, 60 bp, ARTIFICIAL SEQUENCE
  SYNTHETIC DNA
gggtgcgtaaaaatgtacaaattcgatctcaatgattctaaaggtgaagaattattcact

SEQ ID NO: 9 - DNA, 52 bp, ARTIFICIAL SEQUENCE
  SYNTHETIC DNA
tagggcgaattggagctccaccgcggtggcttatttgtacaattcatcccat

SEQ ID NO: 10 - DNA, 60 bp, ARTIFICIAL SEQUENCE; identical to SEQ ID NO: 8
  SYNTHETIC DNA

SEQ ID NO: 11 - DNA, 61 bp, ARTIFICIAL SEQUENCE
  SYNTHETIC DNA
gagtaatagcatctaatggtcaagagttttatcgagacgattatttgtacaattcatccat

SEQ ID NO: 12 - DNA, 27 bp, ARTIFICIAL SEQUENCE
  SYNTHETIC DNA
gccaagcttctccttgacgttaaagta

SEQ ID NO: 13 - DNA, 27 bp, ARTIFICIAL SEQUENCE
  SYNTHETIC DNA
gccggatcctttgtaactgagctgtca

SEQ ID NO: 14 - DNA, 26 bp, ARTIFICIAL SEQUENCE
  SYNTHETIC DNA
caacgaaggatccaataatcgaagcc

SEQ ID NO: 15 - DNA, 28 bp, ARTIFICIAL SEQUENCE
  SYNTHETIC DNA
ggggaattccttcattttatgagttgct

SEQ ID NO: 16 - DNA, 31 bp, ARTIFICIAL SEQUENCE
  SYNTHETIC DNA
caacgaagctttcaataatcgaagcacttgg

SEQ ID NO: 17 - DNA, 31 bp, ARTIFICIAL SEQUENCE
  SYNTHETIC DNA
tttatgagaagctttgggttgatacctttgc

(19) United States
(12) Patent Application Publication
(10) Pub. No.: US 2008/0199864 A1
(43) Pub. Date: Aug. 21, 2008

(54) METHODS FOR CELL SCREENING OF COMPOUNDS CAPABLE OF MODULATING THE ACTIVITY OF UBIQUITIN-LIGASE SCF COMPLEXES AND THEIR USES

(75) Inventors: Dominique Thomas, Gif-sur-Yvette (FR); Regine Barbey, Villebon-sur-Yvette (FR); Astrid Rouillon, Antony (FR); Yolande Kerjan, Fontenay-aux-Roses (FR)

Correspondence Address: OBLON, SPIVAK, MCCLELLAND MAIER & NEUSTADT, P.C., 1940 DUKE STREET, ALEXANDRIA, VA, 22314, US

(73) Assignee: CENTRE NATIONAL DE LA RECHERCHE SCIENTIFIQUE CNRS, Paris Cedex (FR)

(21) Appl. No.: 11/749,933

(22) Filed: May 17, 2007

Related U.S. Application Data
(63) Continuation of application No. 10/148,170, filed on Oct. 9, 2002, now abandoned.

(30) Foreign Application Priority Data
Dec. 1, 1999 (FR) 99/15138

(51) Int. Cl. C12Q 1/68; C12N 15/00; C12N 1/00

(52) U.S. Cl. 435/6; 435/320.1; 435/255.1

(57) ABSTRACT

The invention concerns methods for cell screening of agents capable of modulating the activity of SCF.sup.Met30 complexes comprising the following steps: (i) contacting the product to be tested with a modified yeast strain, including (a) a hybrid sequence comprising a sequence coding for a Met4 protein, in its wild or mutated form, fused in phase with at least a sequence coding for an appropriate marker, said hybrid sequence being expressed under the control of a promoter, active in the yeast and optionally (b) a reporter transcriptional system, consisting of a reporter gene placed under the control of an appropriate operating sequence or an appropriate yeast promoter, (ii) adding methionine and (iii) determining the level of expression and stability of the expressed protein from the hybrid sequence, and their uses. The invention also concerns plasmids and yeast strains capable of being used in said methods.

The subject of the present invention is methods for cell screening of compounds capable of modulating the activity of SCF ubiquitin-ligase complexes and their uses.

The existence of the controlled degradation of proteins has been known for more than thirty years, but the exact molecular mechanisms involved in this process have only been described over the past few years.

In eukaryotic cells, the main pathway for the selective degradation of proteins outside the lysosomes involves a cascade of reactions which lead, in a first instance, to the labeling of the proteins to be destroyed with a polypeptide, consisting of 76 amino acids, called ubiquitin. The addition of several ubiquitin molecules then targets the protein thus modified toward the proteasome, where it is destroyed. This is known as the ubiquitin-proteasome pathway.

The degradation of a protein being essentially an irreversible process, the ubiquitin-proteasome system is recruited by the numerous regulations and signaling pathways whose aim is to modify long term the cellular processes, in particular the developmental pathways, the regulations of the cell cycle and the responses to the presence of pathogenic agents. It is clear that compatibility of such processes with normal cell life requires tight control of the nature of the proteins to be destroyed.

This necessary selectivity of the processes for degrading proteins is achieved by the enzymes which catalyze the addition of the ubiquitin molecules and which are known by the generic name of ubiquitin ligases.

The proteasome is a protein complex, composed of several subunits, which recognizes proteins when they are modified by the attachment of ubiquitin to their lysine residues. This ubiquitylation, prior to the recognition of the target proteins by the proteasome, involves at least three enzymatic complexes, called E1, E2 and E3. E1 catalyzes the activation of ubiquitin by forming a thioester between itself and ubiquitin, which is then transferred to the enzyme E2, a conjugating enzyme. Finally, the E3 ubiquitin ligase facilitates the recognition of the target by E2 or directly transfers the ubiquitin from E2 to the substrate (Hochstrasser M. et al., (1996), Annu. Rev. Genet, 30, 405-439).

Recently, it was shown that an additional conjugation factor, called E4, was necessary (Koegl M. et al., (1999), Cell, 96, 635-644).

Whereas the factor E1 is common to all the degradation pathways and only serves to activate ubiquitin, the selectivity of the complex responsible for ubiquitylation is provided by E3 ubiquitin ligase, which interacts both with E2 and with the substrate (Hershko A. et al., (1983), J. Biol. Chem., 258, 8206-8214).

Two groups of ubiquitin ligases can be distinguished:

isolated E3 proteins, in particular the family of HECT (“Homologous to E6-AP Carboxyl-Terminus”) proteins which possess a carboxy-terminal domain homologous to that of the human E6-AP protein, involved in the formation of a catalytic intermediate with ubiquitin, and

E3 complexes, among which the family of SCF (S: Skp1; C: Cdc53 or cullin; F: protein containing an F-box) ubiquitin-ligase complexes is the most diversified. SCF ligases were discovered in the yeast Saccharomyces cerevisiae (Feldman R. M. R. et al., (1997), Cell, 91, 221-230; Skowyra D. et al., (1999), Science, 284, 662-665; Patton E. E. et al., (1998), Genes Dev., 12, 692-705) and it was recently shown that they in fact exist in all eukaryotic organisms, from fungi to mammals (Koepp D. M. et al., (1999), Cell, 97, 431-434). SCF complexes comprise at least three common subunits, the Skp1 protein, a protein of the cullin family (Cdc53 in yeast and cullin 1 in humans) and the Hrt1 protein (Rbx1 or Rox1). They also comprise modular receptor subunits which confer specificity toward the substrate, and which are proteins containing an F-box (Skowyra D. et al., (1999) reference cited; Patton E. E. et al., (1998), Trends Genet., 14, 236-243). The F domain is a degenerate motif of about 40 amino acids, which allows the protein containing it to interact specifically with Skp1 (Bai C. et al., (1996), Cell, 86, 263-274). The SCF complexes are very closely associated with a particular E2 enzyme, called Cdc34, which recognizes an independent binding site on Cdc53 (Patton E. E. et al., (1998), Genes dev reference cited).

Currently, although more than 15 proteins containing F-boxes have been identified by means of their sequence homology in the yeast genome, only three complexes have been described and characterized in this organism, namely: SCF^{Cdc4}, SCF^{Grr1} and SCF^{Met}³⁰. Each of these complexes has as target one or more specific substrates which will be degraded after ubiquitylation; thus SCF^{Cdc4} has as target the CDK (cyclin-dependent kinase) inhibitors, Sic1p and Far1p, SCF^{Grr1} has as target the G1 cyclins, Cln/Cln2, and SCF^{Met30} has as target the CDK inhibitor, Swep1 (Koepp D. M. et al., (1999), reference cited). The inventors have shown that the SCF^{Met30} complex also plays a role in the negative regulation of the metabolism of sulfur amino acids (Thomas D. et al., (1995), Mol. Cell, Biol., 15, 6526-6534).

Homologs of the SCF^{Met30} complex were recently discovered in other organisms. Thus, a Slimb (Supernumerary Limbs) gene encoding F-box proteins has been identified in drosphila which has as target the inhibitory protein IκB, the β-catenin/Armadillo (β-cat/Arm) trans-criptional coactivator or the Cubitus interruptus (Ci) regulatory protein (Spencer E. et al., (1999), Genes. Dev., 13, 284-294).

In humans, a complex homologous to the SCF^{Met30} complex, the SCF^{β-TrCP} complex, has also been identified. The β-TrCP protein (β-transducing repeat containing protein) has been described, for the first time, in the context of an infection with the HIV-1 virus. It is an F-box protein, induced by the viral Vpu protein (Margottin F. et al., (1998), Mol. Cell., 1, 565-574), which is involved in the degradation of the CD4 cellular receptors present at the surface of infected cells, and is necessary for obtaining infectious HIV virus particles. Additional studies have made it possible to show that, in the absence of infection with the HIV-1 virus, β-TrCP allows specific recognition and the destruction of targets such as IκBα, an inhibitor of the ubiquitous transcription factor NFκB, which directly regulates the immune and inflammatory response, β-catenin, a protein of the Wg/Wnt pathway, whose abnormal solubilization leads to the activation of the transcription of oncogenic genes and which is involved in several types of cancer (Hart M. et al., (1999), Curr. Biol., 9, 207-210; Kroll et al., (1999), J. Biol. Chem., 274, 7941-7945).

More recently, a protein homologous to β-TrCP, the FWD1 protein, was identified in mice (Hatakeyama S. et al., (1999), Proc. Natl. Acad. Sci. USA, 96, 3859-3863).

The SCF complexes in fact constitute the prototypes of an even larger superfamily of ubiquitin ligases, also comprising the APC complexes (anaphase promoting complexes), which control cell division, and the VCB (VHL-EloginC/ElonginB) complexes, in particular those which are involved in certain rare forms of hereditary cancers and those which contain an SOCS (suppressors of cytokine signaling) box.

In most of the signaling pathways which involve the proteasome, the signaling cascade which follows is preserved:

A. activation or inhibition of a protein kinase, under the influence of an extracellular stimulus,

B. phosphorylation (or dephosphorylation) of the target protein,

C. recognition and ubiquitylation of the target protein phosphorylated by the SCF complex, and

D. targeting of the ubiquitinylated protein toward the proteasome where it is finally degraded. The dephosphorylated protein escapes degradation and accumulates, including in the nucleus.

The characterization of the signaling pathways controlled by the SCF complexes, in various eukaryotic organisms, shows their central role in maintaining cellular homeostasis.

The inventors have thus identified, in yeast, the Met30 protein as a factor involved in the transcriptional repression of the genes involved in the biosynthesis of sulfur amino acids (cysteine, methionine and S-adenosyl-methionine (AdoMet)) (Thomas D. et al., (1995), Mol. Cell. Biol., 15, 6526-6534). in yeast, this metabolic pathway has a set of about 25 genes (MET genes) most of which are strictly coregulated, that is to say that in response to an intracellular increase in AdoMet, which may be easily obtained by adding methionine to the yeast growth medium, the transcription of these genes is blocked. In humans, the metabolism of sulfur amino acids is very different because of the fact that, unlike yeast, it is not capable of assimilating sulfate and its growth requires a dietary supply of sulfur amino acids (methionine).

Previous studies have demonstrated the existence of at least five different transcriptional factors in yeast which are necessary for the transcriptional activation of the MET genes; among these factors, there may be mentioned two leucine zipper proteins (bZIP), Met4 and Met28, two zinc finger proteins, Met31 and Met32, and the Cbf1 protein which is also a component of the yeast kinetochore (Thomas D. et al. (1997), Microbiol. Mol. Biol. Rev., 61, 503-532). The Met4 factor in particular does not have a known function homology in humans. Depending on the genes, various combinations of factors combine into complexes which recognize specific sequences upstream of the MET genes; thus the Cbf1-Met4-Met28 complex attaches to the TCACGTC sequence upstream of the MET16 gene whereas the Met4-Met28-Met31 and Met4-Met28-Met32 complexes recognize the AAACTGTG motif upstream of the MET3 and MET28 genes (Kuras L. et al. (1997), EMBO J., 16, 2441-2451; Blaiseau P. L. et al. (1998) EMBO J., 17, 6327-6336). In all these complexes, the transcriptional activation of the various MET genes depends only on a single activation domain carried by the Met4 subunit.

The dysfunction of the ubiquitin-proteasome pathway, a pathway for the degradation of proteins, has been established in numerous pathologies of extremely diverse natures, in particular cancers, genetic diseases, Parkinson's disease, Alzheimer's disease, inflammatory syndromes and viral infections. Thus, it is known that any mutation present on the target proteins around phosphorylation sites abolishes recognition of the mutated proteins by the SCF complexes and leads to their stabilization. It was recently demonstrated that such mutations, which affect β-catenin and prevent its destruction, are involved in tumor transformation in numerous tissues (colon cancer, melanoma, hepatocarcinoma and the like). By contrast, the excessive degradation of β-catenin in the neurons has been implicated in Alzheimer's disease, and is implicated in neuronal death by apoptosis which occurs in this pathology.

Accordingly, the search for molecules, capable of acting on pathologies linked to a dysfunction in the ubiquitin cascade and the degradation of the proteasome, in particular anticancer agents, anti-inflammatory agents and antiviral agents (Maniatis T. (1999) reference cited; F. Margottin et al., (1999), Médecine et Sciences, 15, 1008-1014) has proved highly desirable.

Various methods for screening compounds which are active on the ubiquitin cascade and the degradation of the proteasome have been proposed and use a ubiquitin-ligase/specific substrate pair, preferably of human origin, in which the substrate is a protein regulating a cellular process whose excessive degradation or lack of degradation by the ubiquitin-proteasome pathway is directly involved in the pathology to be treated:

International application PCT WO 97/12962 describes, for screening anticancer agents, a method which uses a ubiquitin-ligase E3 containing a C-terminal region homologous to the catalytic domain of the E6-AP protein (“HECT” family: h-pub1, h-pub2, h-pub3, s-pub1) capable of specifically ubiquitylating a cell cycle regulating protein such as tyrosine phosphatase cdc 25 or the tumor suppressor p53,

International application PCT WO 99/04033 and American patent U.S. Pat. No. 5,932,425, for screening substances capable of treating disorders characterized by an increase in the transcriptional activity of the NF-κB factor (autoimmune diseases, inflammatory conditions, cachexia, AIDS), describe a method which also uses a human ubiquitin-ligase of the HECT family (RSC or KIAAN), capable of specifically ubiquitylating the IκB protein which forms an inactive cytoplasmic complex with the ubiquitous transcription factor NF-κB, which regulates the immune and inflammatory response,

International application PCT WO 99/38969 describes a method which uses an F-box protein (human β-TrCP protein) which binds to the Vpu viral protein, in order to screen anti-HIV agents. Vpu which serves as intermediate in the targeting of CD4 cell receptors toward the ubiquitin-proteasome degradation pathway thus participates in the reduction of the number of functional CD4+ T lymphocytes which is responsible for the immunodeficiency linked to HIV infection.

In general, in these various methods, which use systems of human origin, there is a direct link between the pathology to be treated and the proteins the modulation of whose degradation is sought.

However, continuing their work, the inventors have shown that unexpectedly the SCF^{Met30} complex controls the transcription of all the genes of the sulfate assimilation pathway and that the transcriptional repression of this pathway, in response to an increase in the intracellular AdoMet concentration, results from the specific degradation of Met4 which involves the SCF^{Met30} complex. The existence of this control establishes for the first time the direct link between the activity of the SCF^{Met30} complex and the regulation of the metabolism of sulfur amino acids in yeast.

Taking advantage of the extreme conservation of the molecular mechanisms involved in the ubiquitin-proteasome pathway for degrading proteins in all eukaryotic cells, the inventors have developed a method for the cellular screening of compounds capable of acting on the pathologies linked to the dysfunction of the ubiquitin-proteasome cascade in humans using this pathway in a manner which had never been envisaged. They indeed selected a protein of the ubiquitin-proteasome pathway, whose stability is not directly involved in the pathologies to be treated, but which makes it possible to have very efficient screening tools which have the advantage of being rapid and reliable.

The subject of the present invention is consequently methods for cell screening of compounds or of agents capable of modulating the activity of SCF^{Met30} complexes, characterized in that they comprise the following steps:

(i) bringing the product to be tested into contact with a modified yeast strain containing (a) a hybrid sequence comprising a sequence encoding a Met4 protein, in its wild-type or mutated form, fused in phase with at least one sequence encoding an appropriate marker, said hybrid sequence being expressed under the control of a promoter active in yeast and optionally (b) a reporter transcriptional system consisting of a reporter gene placed under the control of an appropriate operator sequence or of an appropriate yeast promoter,

(ii) adding methionine at repressive concentrations of between 0.03 mM and 20 mM, or at nonrepressive concentrations, and

(iii) determining the level of expression and stability of the protein expressed from the hybrid sequence, either by visualization and/or quantification, or by determination of the activity of the reporter gene.

In an advantageous embodiment of the methods according to the invention, said methods may comprise the following steps in parallel:

(iv) bringing the product to be tested into contact with a modified yeast strain containing a hybrid sequence comprising a sequence encoding a Met30 protein, in its wild-type or mutated form, fused in phase with at least one sequence encoding an appropriate marker, said hybrid sequence being expressed under the control of a promoter active in yeast,

(v) adding methionine at repressive concentrations of between 0.03 mM and 20 mM, or at nonrepressive concentrations, and

(vi) determining the level of expression and stability of the protein expressed from the hybrid sequence, either by visualization and/or quantification, or by determination of the activity of the reporter gene.

In another advantageous embodiment of the methods according to the invention, said methods may comprise, in addition, an additional means using a cellular system of control, which will make it possible to ensure the specificity of response of the hybrid systems or of the transcriptional reporter systems used in steps (i) to (vi), said means comprising the following steps:

(vii) bringing the product to be tested into contact with a modified yeast strain containing a hybrid sequence comprising a sequence encoding a Met28 protein, in its wild-type or mutated form, fused in phase with at least one sequence encoding an appropriate marker, said hybrid sequence being expressed under the control of a promoter active in yeast,

(viii) adding methionine at repressive concentrations of between 0.03 mM and 20 mM, or at nonrepressive concentrations, and

(ix) determining the level of expression and stability of the protein expressed from the hybrid sequence, either by visualization and/or quantification, or by determination of the activity of the reporter gene.

In another advantageous embodiment of the methods according to the invention, said methods may comprise, in addition, an additional means using an acellular system of control which is based on measuring the levels of transcription of the hybrid sequences and of the metabolic genes MET16 and MET25, said means comprising the following steps:

(x) extracting the total RNAs of the modified strains used either in step (i), or in step (iv), or in step (vii) and

(xi) measuring the levels of transcription of the hybrid sequence and that of the metabolic genes MET16 and MET25.

The subject of the present invention is also methods for cell screening of compounds capable of modulating the activity of the SCF^{Met30} complexes, characterized in that they comprise the following steps:

(xii) bringing the product to be tested into contact with a modified yeast strain containing a reporter transcriptional system consisting of a reporter gene placed under the control of an appropriate yeast promoter,

(xiii) adding methionine at repressive concentrations of between 0.03 mM and 20 mM, or at nonrepressive concentrations, and

(xiv) determining the activity of the reporter gene.

For the purposes of the present invention, mutated form of a protein is understood to mean a protein modified either by insertion, deletion or substitution of one or more amino acids.

For the purposes of the present invention, methionine is understood to mean the (L) form or the (DL) form of methionine.

For the purposes of the present invention, the expression appropriate operator sequence controlling the reporter gene is understood to mean a sequence recognized by a DNA-binding factor fused in phase with a Met4 protein, such as the LexA-Met4 fusion protein.

For the purposes of the present invention, the expression yeast promoter controlling the reporter gene is understood to mean a promoter activated by the Met4 transcription factor, such as a MET gene such as MET3, MET10, MET14, MET16, MET25 or MET28.

In an advantageous embodiment of the methods according to the invention, the marker used for the construction of the hybrid sequence is chosen from the group consisting of: the antigenic peptides, for example the hemagglutinin (Ha) antigenic unit, the intrinsic fluorescence proteins, such as for example the green fluorescence protein (Green Fluorescence Protein or GFP), the proteins with measurable enzymatic activity, and the DNA-binding factors such as for example E. coli LexA.

In another advantageous embodiment of the methods according to the invention, the promoter allowing the expression of the hybrid protein may be either an inducible promoter active in S. cerevisiae, which may be advantageously chosen from the group consisting of the promoter of the GAL1 gene and the promoter of the CUP1 gene, or a constitutive promoter, such as for example the promoter of the S. cerevisiae ADH1 gene.

In another advantageous embodiment of said methods, the reporter gene is chosen from the group consisting of the reporter genes whose activity can be visualized by a colorimetric method, such as for example the P. putida XylE gene and the E. coli LacZ gene, and the metabolic genes, whose activity can be measured by a growth test, such as the S. cerevisiae HIS3, URA3, TRP1 and LEU2 genes.

Said metabolic genes may be alternatively and advantageously used as genes for selecting modified yeasts containing a hybrid sequence and/or a reporter transcriptional system, as defined above.

In another advantageous embodiment of said methods, said reporter gene (present in the reporter transcriptional system) is placed under the control:

either of the promoter of a MET gene, preferably MET3, MET10, MET14, MET16, MET25 or MET28,

or of an operator sequence recognized by the LexA-Met4 fusion protein (LexA operators).

In another advantageous embodiment of the methods according to the invention, the yeast strains carry one or more mutations which increase permeability to the products to be tested (Vidal M et al., (1999), Trends in Biotechnol., 17, 374-381).

To carry out the methods according to the invention, it is possible to use yeast strains possessing the genetic background of the W303 strain of the S. cerevisiae yeast which is described by Bailis A. M. et al., (Genetics, (1990), 126, 535-547) or any other strain characterized by said yeast.

The transformation of the yeast cells by exogenous DNA was carried out using techniques known to persons skilled in the art, in particular the technique described by Schiestl R. H. et al. (Curr. Genet., (1989), 16, 339-346), genetic techniques (sporulation, dissection and evaluation of markers, and the like) are also known, and there may be cited in particular those described by Sherman F. et al. (in Methods in Yeast Genetics: a Laboratory Manual, (1979), Cold Spring Harbor, N.Y.) and the reverse genetic techniques described by Rothstein R. (Methods in Enzymology, (1991), 194, 281-301); as a technique for integration directed at the locus by single homologous recombination (crossing over), there may be mentioned that which is described in Orr-Weaver et al. (1983; reference cited).

In accordance with the invention, the yeasts may be transformed with plasmids constructed by conventional molecular biology techniques, in particular according to the protocols described by Sambrook J. et al. (Molecular cloning Laboratory Manual, 2nd edition, (1989), Cold Spring Harbor, N.Y.) and Ausubel F. M. et al. (Current Protocols in Molecular Biology, (1990-1999), John Wiley and Sons, Inc. New York).

In accordance with the invention, the activity of the reporter genes and of the MET genes is measured, according to the reporter gene and the promoter used, by techniques known per se, in particular calorimetric techniques, enzymatic techniques, immunological techniques, fluorescence techniques or techniques for selection on an appropriate growth medium.

In accordance with the invention, the activity of the SCR^{Met30} complex is determined by measuring the level of expression and of stability of the protein encoded by the hybrid sequence and/or by the activity of the transcriptional reporter system; indeed, the hybrid protein encoded by said hybrid sequence advantageously preserves the intrinsic properties of each of the two fused elements constituting it. For example, the Met4 marker protein is selectively degraded (by addition of methionine to the culture medium) by the ubiquitin-proteasome pathway (property of Met4 protein) and is visualized, in accordance with the associated marker: when the marker present in the hybrid protein is the GFP protein, then the activity of the SCF^{Met30} complex is visualized by observing and by quantifying the fluorescence of the GFP-Met4 or GFP-Met30 hybrid protein; when the marker present in the hybrid protein is the hemagglutinin (Ha) antigenic unit, then the activity of the SCF^{Met30} complex is visualized by immunological techniques, such as protein transfer techniques (Western Blotting), ELISA techniques or immunoprecipitation techniques (Harlow E. et al., (1988), Antibodies, a Laboratory Manual, Cold Spring Harbor, N.Y.); when the cells contain a transcriptional reporter system containing the Xy1E gene, then the activity of the SCF^{Met30} complex is visualized by vaporization, on yeast cells, of catechol at a concentration of between 50 mM and 1 M, and by measuring the appearance of a yellow color (Worsay M. J. et al., (1975), J. Bacteriol., 124, 7-13); when the cells contain a transcriptional reporter system containing the E. coli LacZ gene, then the activity of the SCF^{Met30} complex is visualized by measuring the appearance of a blue color, on a medium containing the colorigenic substrate X-Gal (Sambrook J. (1989), reference cited); when the cells contain a transcriptional reporter system containing the S. cerevisiae HIS3 gene, then the activity of the SCF^{Met30} complex is visualized by growing the yeast on a minimum medium not containing histidine, in the presence of aminotriazole, at concentrations of 0.5 mM to 200 mM.

Because of the existence of numerous subunits common to the various SCF complexes, said subunits being in stoichiometric equilibrium, the SCF^{Met30} complex may serve as a model for screening molecules for their capacity to modulate either the activity of the signaling pathways controlled by the SCF complexes as a whole, or that of the ubiquitin-proteasome pathway, a pathway for the degradation of proteins.

Surprisingly, this second system (ubiquitin-proteasome pathway), as used in the present invention, by using, as substrate, the Met4 protein, is particularly advantageous for screening molecules or agents capable of acting on pathologies linked to the dysfunction of the ubiquitin-proteasome cascade in humans such as cancers, genetic diseases, Parkinson's disease, Alzheimer's disease, inflammatory syndromes and viral infections:

simplicity: the induction of the SCF^{Met}³⁰/Met4 complex system is carried out simply by adding methionine to the growth medium, and when the marker is the GFP protein, the activity of the SCF^{Met30} complex is visualized directly by observing and/or quantifying the fluorescence emitted,

speed of development: the reverse genetic techniques, the integral knowledge of the genome of the yeast and the molecular biology methods adapted to this organism ensure a rapid use of indicator stable strains,

speed of growth and of screening: yeast is a rapidly growing and high yield microorganism, which allows the production of modified cells for a large number of screenings,

low cost: yeast is a microorganism whose culture, storage and characterization are not very expensive.

The methods of screening according to the invention may serve in particular to select active agents such as anticancer agents, anti-inflammatory agents, antiviral agents or agents active in genetic diseases, in particular in Parkinson's disease and Alzheimer's disease.

For the purposes of the present invention, compound or agent is understood to mean any molecule derived from methods of syntheses or natural resources.

The subject of the present invention is also the use of agents selected by the methods according to the present invention, for the preparation of medicaments intended for the treatment of diseases linked to disorders of the activity of the SCF complexes or of the ubiquitin-proteasome pathway, such as cancers, genetic diseases, Parkinson's disease, Alzheimer's disease, inflammatory syndromes and viral infections.

The subject of the present invention is also plasmids, characterized in that they contain a hybrid sequence comprising a sequence encoding a Met4, Met28 or Met30 protein in its wild-type or mutated form, fused in phase with at least one sequence encoding a marker chosen from the Met protein, from the group consisting of: antigenic peptides, intrinsic fluorescence proteins and proteins with measurable enzymatic activity, it being possible for said hybrid sequence to be expressed in yeast under the control of a constitutive or inducible promoter.

In accordance with the invention:

when said plasmid comprises a sequence encoding a Met4 protein, then said marker is chosen from antigenic peptides, intrinsic fluorescence proteins,

when said plasmid comprises a sequence encoding a Met28 protein, then said marker is chosen from antigenic peptides, intrinsic fluorescence proteins and proteins with measurable enzymatic activity, and

when said plasmid comprises a sequence encoding a Met30 protein, then said marker is chosen from intrinsic fluorescence proteins and proteins with measurable enzymatic activity.

According to an advantageous embodiment of said plasmids, they are selected from the group consisting of:

plasmids containing a sequence encoding a Met4 or Met28 protein fused with a peptide comprising three hemagglutinin (HA) antigenic units, said sequence being expressed under the control of the GAL1 inducible promoter,

plasmids containing a sequence encoding a Met4, Met28 or Met30 protein, fused with a GFP protein, said sequence being respectively expressed under the control of the constitutive promoters MET4, MET28 or MET30 or under the control of the GAL1 inducible promoter,

plasmids containing a sequence encoding a Met30 protein, fused with a GFP protein and with a peptide comprising 3 hemagglutinin (HA) antigenic units, said sequence being expressed under the control of the GAL1 promoter.

The subject of the present invention is also plasmids, characterized in that they contain a hybrid sequence comprising a sequence encoding a Met4 protein, in its wild-type or mutated form, fused in phase with at least one sequence encoding the DNA-binding factor LexA, and the TRP1 gene or the LEU2 gene as genes for selecting yeasts modified with said plasmids.

The subject of the present invention is also plasmids containing a reporter transcriptional system consisting of a reporter gene placed under the control either of an appropriate yeast promoter or of an appropriate operator sequence, as defined above.

Advantageously, said plasmids contain the LacZ or XylE reporter gene, expressed under the control, either of the MET16 promoter, or of LexA operators.

The subject of the present invention is also yeast strains, characterized in that they are stably modified with at least one plasmid according to the present invention.

Other characteristics and advantages of the invention appear in the remainder of the description and the examples, which are illustrated by figures in which:

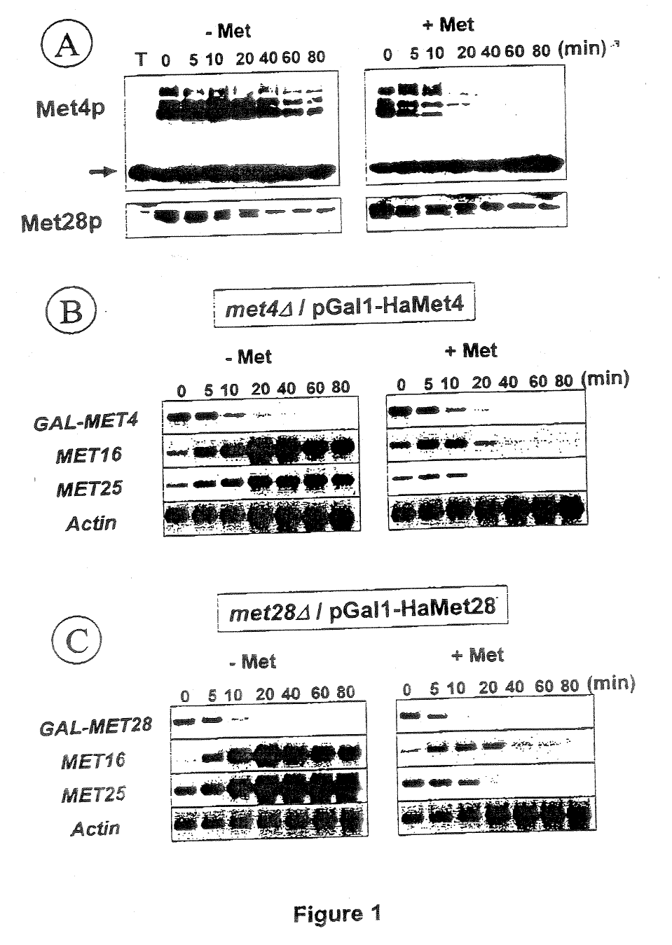

FIG. 1 illustrates the influence of the addition of methionine at repressive concentrations to the culture medium (A) the cells containing a plasmid coding the proteins labeled with hemagglutinin (Ha) antigenic units under the control of the GAL1 promoter, and prepared according to the procedure described in examples 2 and 4, are cultured, according to the procedure described in example 12, in a minimum medium containing 2% galactose for 90 minutes, or in the presence of methionine at a repressive concentration (+Met), or in the absence of methionine (−Met); (B) and (C) the total RNAs are extracted, according to the procedure described in example 12 from cells used in (A) and expressing either the Ha-Met4 hybrid protein, or the Ha-Met28 hybrid protein. The cells are cultured under the conditions described in (A) and are analyzed with MET4, MET16, MET25, MET28. “met4Δ” corresponds to a W303 cell modified with a chromosomal copy of the inactivated MET4 gene; “met28Δ” corresponds to a W303 cell modified with a chromosomal copy of the inactivated MET28 gene.

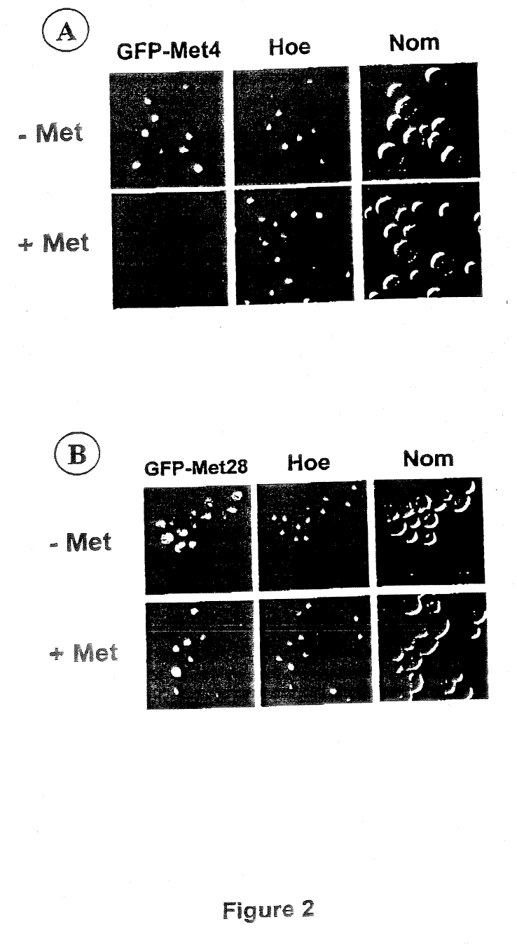

FIG. 2 illustrates the location of the GFP-Met4 and GFP-Met28 hybrid proteins in wild-type cells, in the absence of methionine (−Met) or in the presence of methionine at a repressive concentration (+Met). The cells are cultured under the conditions described in example 12. “Hoe” corresponds to the colored indicator specific to the nuclei, Hoechst 333-42; “Nom” corresponds to the image obtained by Nomarski interference microscopy.

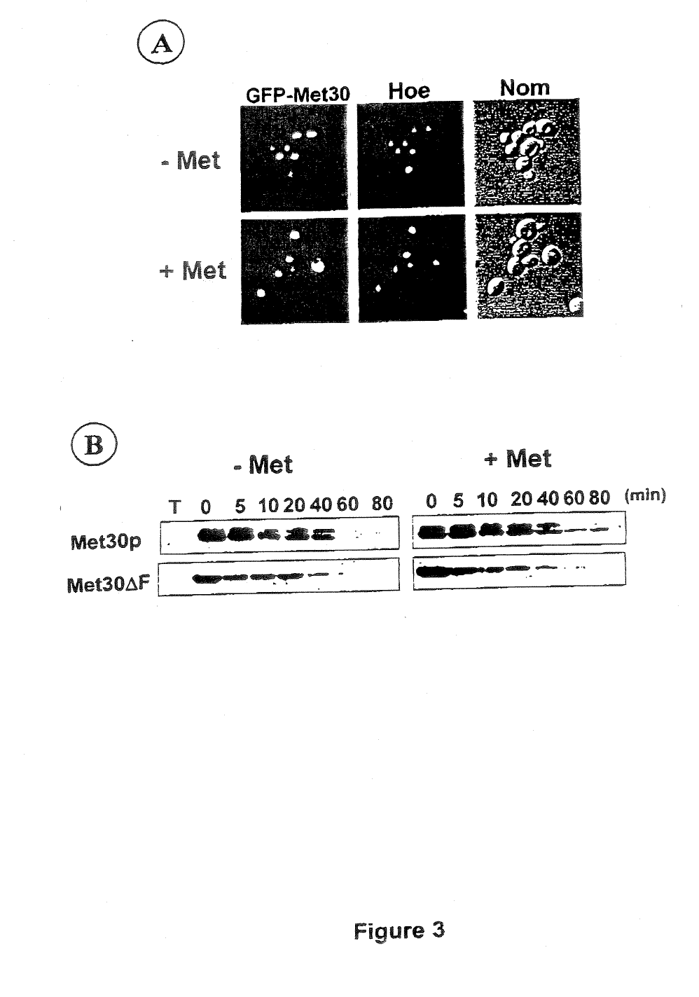

FIG. 3 illustrates (A) the location of the GFP-Met30 hybrid protein in wild-type cells, in the absence of methionine (−Met) or in the presence of methionine at a repressive concentration (+Met). The cells are cultured under the conditions described in example 12. “Hoe” corresponds to the colored indicator Hoechst 333-42; “Nom” corresponds to the image obtained by Nomarski interference microscopy; (B) stability of the Ha-Met30 and Ha-Met30ΔF hybrid proteins in wild-type W303-1A strains.

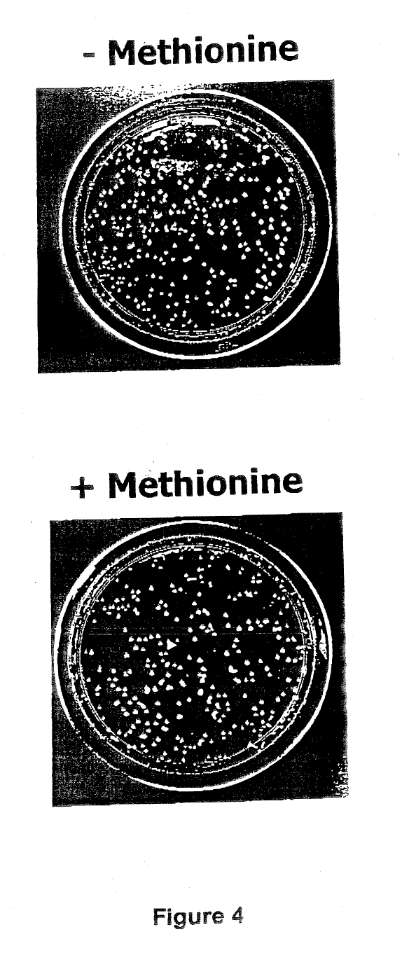

FIG. 4 illustrates the activity of the LexAopXylE reporter gene in the absence of methionine (−Met) or in the presence of methionine at a repressive concentration, from a yeast strain (C190) expressing the hybrid protein encoded by the plasmid pLexMet4-7; the visualization is carried out by measuring catechol oxidase.

### EXAMPLE 1

### Construction of the Basic Plasmids pGal316Flu, pGal306Flu and pFL39Flu

1.1. Construction of the Plasmids pGal316Flu and pGal306Flu

1.1.1. Construction of the Plasmids pGal316 and pGal306:

A fragment of 900 base pairs (bp) of the plasmid pJN1 (Nehlin J. et al., (1990) EMBO J., 9, 2891-2898) containing a fusion between the promoters of the S. cerevisiae genes, GAL1 and TPK2, is amplified by PCR (polymerase chain reaction), using the oligonucleotides “olGal10” having the sequence SEQ ID No. 1: 5′CAAAGAAGCTTAATAATCATATT3′ and “olGalTPK” having the sequence SEQ ID No. 2: 5′TTGACCAACTGGCTGAGCC3′.

The fragment obtained is digested with the restriction enzymes HindIII and EcoRI and inserted:

(i) into the S. cerevisiae-E. coli shuttle plasmid pRS306 whose sequence has been deposited in the EMBL databank, with the identifier “PRS316”, under the No. U03442, digested beforehand with the enzymes HindIII and EcoRI, producing the plasmid pGal316.

(ii) into the S. cerevisiae-E. coli shuttle plasmid pRS306 whose sequence has been deposited in the EMBL databank, with the identifier “PRS306”, under the No. U03438, digested beforehand with the restriction enzymes HindIII and EcoRI.

The plasmid pGal306 is thus produced.

1.1.2. Construction of the plasmids pGal316Flu and pGal306Flu:

A double-stranded DNA fragment corresponding to the sequence SEQ ID No. 3 and encoding a repetition of 3 hemagglutinin (Ha) antigenic units is inserted:

(i) into the plasmid pGal316 obtained at point 1.1.1., digested beforehand with the restriction enzyme EcoRI and whose ends have been made blunt by treatment with the Klenow fragment of E. coli DNA polymerase, producing the plasmid pGal316Flu,

(ii) into the plasmid pGal306 prepared at point 1.1.1., digested beforehand with the restriction enzyme EcoRI and whose ends have been made blunt by treatment with the Klenow fragment of E. coli DNA polymerase, producing the plasmid pGal306Flu.

1.2. Construction of the Plasmid pFL39Flu:

In a first stage, the EcoRI site present in the polylinker of the S. cerevisiae-E. coli shuttle plasmid pFL39 was destroyed. For that, the plasmid pFL39, whose sequence is that deposited in the EMBL databank, with the identifier “CVPFL39”, under the No. X70483, was digested with EcoRI, the ends made blunt by treating with the Klenow fragment of E. coli DNA polymerase, and the product thus treated was self-ligated, producing the plasmid pFL39E0.

In a second stage, the HindIII-PstI fragment of the plasmid pGal316Flu prepared according to the procedure described at point 1.1. and comprising the GAL1 promoter region and the region encoding the Ha antigenic region was inserted into the plasmid pFL39E0 digested beforehand with the HindIII and PstI enzymes.

The plasmid pFL39Flu is thus obtained.

### EXAMPLE 2

### Plasmids Allowing the Expression in Yeast of Ha-Met4 Hybrid Proteins Under the Control of the GAL1 Promoter.

The plasmids which follow allow the expression, in the S. cerevisiae yeast, of derivatives of the Met4 protein comprising a repetition of three hemagglutinin (Ha) antigenic units at their amino-terminal end.

2.1. Construction of the Plasmid pGal316FluMet4:

The EcoRI-BamHI DNA fragment of the vector pLexM4-1 (Thomas D. et al., (1992), Mol. Cell. Biol., 12, 1719-1727) encoding amino acids 15 to 666 of the Met4 protein was cloned into the plasmid pGal316Flu digested beforehand with the enzymes EcoRI and BamHI, producing the plasmid pGal316FluMet4. This plasmid can replicate autonomously in yeast.

2.2. Construction of the Plasmid pGal316FluMet4Δ12:

The EcoRI-XbaI DNA fragment of the vector pLexM4Δ12 (Kuras L. et al., (1995), Mol. Cell. Biol., 15, 208-216) encoding a derivative comprising amino acids 15 to 79 and 180 to 666 of the Met4 protein was cloned into the plasmid pGal316Flu digested beforehand with the enzymes EcoRI and XbaI, producing the plasmid pGal316FluMet4Δ12. This plasmid can replicate autonomously in yeast.

2.3. Construction of the Plasmid pGal316FluMet4Δ30:

The EcoRI-XbaI DNA fragment of the vector pLexM4×30 (Kuras L. et al., (1995) reference cited) encoding a derivative comprising amino acids 15 to 211 and 232 to 666 of the Met4 protein was cloned into the plasmid pGal316Flu digested beforehand with the enzymes EcoRI and XbaI, producing the plasmid pGal316FluMet4×30. This plasmid can replicate autonomously in yeast.

2.4. Construction of the Plasmid pGal316FluMet4Δ37:

The EcoRI-XbaI DNA fragment of the vector pLexM4Δ37 (Kuras L. et al., (1995), reference cited) encoding a derivative comprising amino acids 15 to 344 and 366 to 666 of the Met4 protein was cloned into the plasmid pGal316Flu digested beforehand with the enzymes EcoRI and XbaI, producing the plasmid pGal316FluMet4Δ37. This plasmid can replicate autonomously in yeast.

2.5. Construction of the Plasmid pGal316FluMet4ΔLZ:

The EcoRI-XbaI DNA fragment of the vector pLexM4-3 (Thomas D. et al., (1992), reference cited) encoding amino acids 15 to 616 of the Met4 protein was cloned into the plasmid pGal316Flu digested beforehand with the enzymes EcoRI and XbaI, producing the plasmid pGal316FluMet4ΔLZ. This plasmid can replicate autonomously in yeast.

2.6. Construction of the Plasmid pFL39FluMet4:

The EcoRI-BamHI DNA fragment of the vector pLexM4-1 (Thomas D. et al., (1992), reference cited) encoding amino acids 15 to 666 of the Met4 protein was cloned into the plasmid pFL39Flu digested beforehand with the enzymes EcoRI and BamI, producing the plasmid pFL39FluMet4. This plasmid can replicate autonomously in yeast.

### EXAMPLE 3

### Construction of the Plasmids Allowing the Expression in Yeast of GFP-Met4 Hybrid Proteins

The plasmids which follow allow the expression in the S. cerevisiae yeast of Met4 protein derivatives fused with the Aequora victoria green fluorescence protein (GFP).

3.1 Construction of the Plasmid pGFPMet4

A fragment of 710 base pairs (bp) of the plasmid pGFPmut3, encoding the GFP3 protein (Green Fluorescent Protein mut3, product of the Aequora victoria GFP gene whose sequence has been deposited at the EMBL bank, with the identifier “AVU73901”, under the No. U73901) was amplified by PCR (polymerase chain reaction) using the oligonucleotides “olGFPM4-5” having the sequence SEQ ID No. 4: 5′ACGCGAATTCATGTCTAAAGGTGAATTA3′ and “olGFPM4-3” having the sequence SEQ ID No. 5: 5′ACGCGAATTCTTTGTACAATTCATCCAT3′.

The fragment obtained was digested with the restriction enzyme EcoRI and inserted into the plasmid pM4-5 (Kuras L. et al., (1995), reference cited) digested beforehand with the enzyme EcoRI, producing the plasmid pGFPMet4.

This plasmid allows the expression, under the control of the MET4 promoter, of a GFP-Met4 hybrid protein comprising amino acids 15 to 666 of Met4, and can replicate autonomously in yeast.

3.2. Construction of the Plasmid pGal316GFPMet4

The EcoRI-BamHI fragment of the plasmid pGFPMet4, encoding the GFP-Met4 fusion, was inserted into the plasmid pGal316 digested beforehand with the enzymes EcoRI and BamHI, producing the plasmid pGal316GFPMet4. This plasmid can replicate autonomously in yeast.

3.3. Construction of the Plasmid pGal316GFPMet4Δ12:

The EcoRI-EcoRI DNA fragment of the vector pGal316GFPMet4 prepared according to the procedure described above and encoding GFP was cloned in phase into the plasmid pGal316FluMet412 digested beforehand with the enzyme EcoRI, producing the plasmid pGal316GFPMet4Δ12. This plasmid can replicate autonomously in yeast.

3.4. Construction of the Plasmid pGal316GFPMet4Δ30:

The EcoRI-EcoRI DNA fragment of the vector pGal316GFPMet4 prepared according to the procedure described at point 3.2 and encoding GFP was cloned in phase into the plasmid pGal316FluMet4Δ30 digested beforehand with the enzyme EcoRI, producing the plasmid pGal316GFPMet4Δ30. This plasmid can replicate autonomously in yeast.

3.5. Construction of the Plasmid pGal316GFPMet4Δ37:

The EcoRI-EcoRI DNA fragment of the vector pGal316GFPMet4 prepared according to the procedure described at point 3.2 and encoding GFP was cloned in phase into the plasmid pGal316FluMet4Δ37 digested beforehand with the enzyme EcoRI, producing the plasmid pGal316GFPMet4Δ37. This plasmid can replicate autonomously in yeast.

3.6. Construction of the Plasmid pGal306GFPMet4:

The Not1-Asp718 DNA fragment of the vector pGal316GFPMet4 prepared according to the procedure described at point 3.2 and comprising the entire GAL1 promoter contained in the sequence deposited at the EMBL databank under the identifier “SCGAL10”, under the No. K02115, and the GFP-Met4 fusion (residues 15 to 666 of Met4) was inserted into the plasmid pRS306 digested beforehand with the Not1-Asp718 enzymes, producing the plasmid pGal306GFPMet4.

3.7. Construction of the Plasmid pGal306GFPMet4Δ12:

The Not1-Asp718 DNA fragment of the vector pGal316GFPMet4Δ12 prepared according to the procedure described at point 3.3 and comprising the entire GAL1 promoter and the GFP-Met4Δ12 fusion (residues 1579 and 180-666 of Met4) was inserted into the plasmid pRS306 digested beforehand with the Not1-Asp718 enzymes, producing the plasmid pGal306GFPMet4Δ12.

3.8. Construction of the Plasmid pGal306GFPMet4Δ30:

The Not1-Asp718 DNA fragment of the vector pGal316GFPMet4Δ30 prepared according to the procedure described at point 3.4 and comprising the entire GAL1 promoter and the GFP-Met4Δ30 fusion (residues 15 to 211 and 221 to 666 of Met4) was inserted into the plasmid pRS306 digested beforehand with the Not1-Asp718 enzymes, producing the plasmid pGal306GFPMet4Δ30.

3.9. Construction of the Plasmid pGal306GFPMet4Δ37:

The Not1-Asp718 DNA fragment of the vector pGal316GFPMet4Δ37 prepared according to the procedure described at point 3.5 and comprising the entire GAL1 promoter and the GFP-Met4Δ37 fusion (residues 15 to 352 and 366 to 666 of Met4) was inserted into the plasmid pRS306 digested beforehand with the Not1-Asp718 enzymes, producing the plasmid pGal306GFPMet4Δ37.

### EXAMPLE 4

### Construction of the Plasmids Allowing the Expression in Yeast of Ha-Met28 and GFP-Met28 Hybrid Proteins

4.1. Construction of the Plasmid pGal316FluMet28:

The EcoRI-BamHI DNA fragment of the vector pLexM28-2 (Kuras L. et al., (1996), EMBO J., 15, 2519-2529) encoding amino acids 1 to 166 of the Met28 protein was cloned into the plasmid pGal316Flu digested beforehand with the enzymes EcoRI and BamHI, producing the plasmid pGal316FluMet28. This plasmid can replicate autonomously in yeast. It allows the expression in yeast of a full-length Met28 protein comprising, at its amino-terminal end, a repetition of 3 hemagglutinin (Ha) antigenic units. The Ha-Met28 hybrid protein is expressed under the control of the GAL1 promoter.

4.2. Construction of the Plasmid pDFL39FluMet28:

The EcoRI-BglII DNA fragment of the vector pLexM28-2 (Kuras L. et al., (1996), reference cited) encoding amino acids 1 to 166 of the Met28 protein was cloned into the plasmid pFL39Flu digested beforehand with the enzymes EcoRT and BglII, producing the plasmid pFL39FluMet28. This plasmid can replicate autonomously in yeast. It allows the expression in yeast of a full-length Met28 protein comprising, at its amino-terminal end, a repetition of 3 hemagglutinin (Ha) antigenic units. The Ha-Met28 hybrid protein is expressed under the control of the GAL1 promoter.

4.3. Construction of the Plasmid pGFPMet28:

4.3.1. Construction of the plasmid p314Met28:

The XbaI-HpaI DNA fragment of the vector pMet28-1 (Kuras L. et al., (1996), reference cited) containing the MET28 gene, whose sequence is that deposited at the EMBL databank, under the identifier “SC17015”, under the No. U17015 and its promoter region was isolated, its ends made blunt by treating with the Klenow fragment of E. coli DNA polymerase and this DNA fragment was inserted into the plasmid pRS314 (Sikorski R. S. et al., (1989), Genetics, 122, 19-27) digested beforehand with the enzyme SmaI, producing the plasmid p314Met28. This plasmid can replicate autonomously in yeast.

4.3.2. Construction of the Plasmid p314GFPMet28:

A fragment of 710 base pairs (bp) of the plasmid pGFPmut3, encoding the GFP3 protein, was amplified by PCR using the oligonucleotides “olM28GFP5” having the sequence SEQ ID No. 6:

5′TGAAATTGTTGAATGACATTAAGAGACGGAACATGGGCAGGTCTAAAG
GTGAAGAATTATTC3′

and “olM28GFP3” having the sequence SEQ ID No. 7:

5′AGTCTGTGGAATATAAACGGTCCTTGATCAATGCGGAGTGTTATTTGT
ACAATTCATCCAT3′.

The fragment obtained was inserted into the plasmid p314Met28 by the “gap repair” method according to the technique described by Orr-Weaver et al. (Methods in Enzymology, (1983), 101, 228-245).

Thus, the fragment obtained after PCR and the plasmid p314Met28, digested beforehand with the enzyme BglII, were cotransformed into the yeast strain W303-1A (Bailis A. M. et al., (1990), reference cited), and the clones prototrophic for tryptophan were selected. The plasmid DNA contained in these clones was extracted, transformed into E. coli and the recombinant plasmid p314Met28GFP identified and characterized by enzymatic digestions and sequence. This plasmid allows the expression of a full-length Met28 protein fused at its carboxyl-terminal end with the GFP protein. The Met28-GFP hybrid protein is expressed under the control of the MET28 promoter. This plasmid can replicate autonomously in yeast.

### EXAMPLE 5

### Construction of the Plasmids Allowing the Expression in Yeast of Ha-Met30 and GFP-Met30 Hybrid Proteins

5.1. Construction of the Plasmid pGal316FluMet30ΔN:

The EcoRI-BglII DNA fragment of the vector pDGadMet30-1 (Thomas D. et al. reference cited) encoding amino acids 158 to 640 of the Met30 protein was cloned into the plasmid pGal316Flu digested beforehand with the enzymes EcoRI and BamHI, producing the plasmid pGal316FluMet30ΔN. This plasmid can replicate autonomously in yeast. It allows the expression in yeast of a Met30 protein truncated of its amino-terminal portion but comprising at this end a repetition of 3 hemagglutinin (Ha) antigenic units. The Ha-Met30 hybrid protein is expressed under the control of the GAL1 promoter.

5.2. Construction of the Plasmid pGal316FluMet30:

The EcoRI-EcoRI DNA fragment of the vector pLexMet30-4 (Thomas D. et al., (1995), reference cited) encoding the first 157 amino acids of the Met30 protein was cloned into the plasmid pGal316FluMet30ΔN digested beforehand with the enzyme EcoRI, producing the plasmid pGal316FluMet30. This plasmid can replicate autonomously in yeast. It allows the expression in yeast of a full-length Met30 protein comprising, at its amino-terminal end, a repetition of 3 hemagglutinin (Ha) antigenic units. The Ha-Met30 hybrid protein is expressed under the control of the GAL1 promoter.

5.3. Construction of the Plasmid pFL39FluMet30ΔN:

The EcoRI-BglII DNA fragment of the vector pGadMet30-1 (Thomas D. et al., (1995), reference cited) encoding amino acids 158 to 640 of the Met30 protein was cloned into the plasmid pFL39Flu digested beforehand with the enzymes EcoRI and BamHI, producing the plasmid pFL39FluMet30ΔN. This plasmid can replicate autonomously in yeast. It allows the expression in yeast of a protein of a protein Met30 truncated of its amino-terminal portion but comprising at this end a repetition of 3 hemagglutinin (Ha) antigenic units. The Ha-Met30 hybrid protein is expressed under the control of the GAL1 promoter.

5.4. Construction of the Plasmid pFL39FluMet30ΔF:

This plasmid is constructed according to the procedure described at point 5.3, but expresses a mutated Met30 protein which does not comprise amino acids 187 to 201.

5.5. Construction of the Plasmid pFL39FluMet30

The EcoRI-EcoRI DNA fragment of the vector pLexMet30-4 (Thomas D. et al., (1995), reference cited) encoding the first 157 amino acids of the Met30 protein was cloned into the plasmid pFL39FluMet30ΔN digested beforehand with the enzyme EcoRI, producing the plasmid pFL39FluMet30. This plasmid can replicate autonomously in yeast. It allows the expression in yeast of a full-length Met30 protein comprising, at its amino-terminal end, a repetition of 3 hemagglutinin (Ha) antigenic units. The Ha-Met30 hybrid protein is expressed under the control of the GAL1 promoter.

5.6. Construction of the Plasmid p316FluMet30GFP:

A fragment of 710 base pairs (bp) of the plasmid pGFPmut3, encoding the GFP3 protein, was amplified by PCR using the oligonucleotides “olM30GFP5” having the sequence SEQ ID No. 8:

5′GGGTGCGTAAAAATGTACAAATTCGATCTCAATGATTCTAAAGGTGAA
GAATTATTCACT3′

and “ol316GFP3” having the sequence SEQ ID No. 9:

5′TAGGGCGAATTGGAGCTCCACCGCGGTGGCTTATTTGTACAATTCATC
CCAT3′.

The fragment obtained was inserted into the plasmid pGal316FluMet30 by the “gap repair” method (Orr-Weaver et al., 1983; reference already cited).

Thus, the fragment obtained after PCR and the plasmid pGal316FluMet30, digested beforehand with the enzyme XbaI, were cotransformed into the yeast strain W303-1A and the clones prototrophic for uracil were selected. The plasmid DNA contained in these clones was extracted, transformed into E. coli and the recombinant plasmid p316FluMet30GFP identified and characterized by enzymatic digestions and sequence. This plasmid allows the expression of a full-length Met30 protein, fused at its carboxyl-terminal end with the GFP protein and at its amino-terminal end with a repetition of 3 hemagglutinin (Ha) antigenic units. The Ha-Met30-GFP hybrid protein is expressed under the control of the GAL1 promoter. This plasmid can replicate autonomously in yeast.

5.7. Construction of the Plasmid pMet30GFP:

5.7.1. Construction of the plasmid pMet30-8:

The XbaI-SalI DNA fragment of the vector pMet30-1 (Thomas D. et al., (1995), reference cited) containing the MET30 gene whose sequence is that deposited at the EMBL databank, under the identifier “SCMET30A”, under the No. L26505 and its promoter region was isolated and inserted into the plasmid pRS316 digested beforehand with the enzymes XbaI and SalI, producing the plasmid pMet30-8. This plasmid can replicate autonomously in yeast.

5.7.2. Construction of the Plasmid pMet30GFP:

A fragment of 710 base pairs (bp) of the plasmid pGFPmut3, encoding the GFP3 protein, was amplified by PCR using the oligonucleotides “olM30GFP5” having the sequence SEQ ID No. 10:

5′GGGTGCGTAAAAATGTACAAATTCGATCTCAATGATTCTAAAGGTGAA
GAATTATTCACT3′

and “olM30GFP3” having the sequence SEQ ID No. 11:

5′GAGTAATAGCATCTAATGGTCAAGAGTTTTATCGAGACGATTATTTGT
ACAATTCATCCAT3′.

The fragment obtained was inserted into the plasmid pMet30-8 by the “gap repair” method (Orr-Weaver et al., 1983; reference already cited).

Thus, the fragment obtained after PCR and the plasmid pMet30-8, digested beforehand with the enzyme NheI, were cotransformed into the yeast strain W303-1A and the clones prototrophic for uracil were selected.

The plasmid DNA contained in these clones was extracted, transformed into E. coli and the recombinant plasmid pMet30GFP identified and characterized by enzymatic digestions and sequence. This plasmid allows the expression of a full-length Met30 protein, fused at its carboxyl-terminal end, with the GFP protein. The Met30-GFP hybrid protein is expressed under the control of the MET30 promoter. This plasmid can replicate autonomously in yeast.

### EXAMPLE 6

### Construction of the Plasmids pYi39XyLex, pYi44XyLex, pLexM4-7, pLexM4Δ239, pM16Z1 and pM16Xyl1 which Make it Possible to Visualize the Transcriptional Activity of the Yeast MET Genes

6.1. Construction of the Plasmid pYi39XyLex:

The integration of this plasmid into the yeast genome at the TRP1 locus can be directed by linearizing this plasmid with the enzyme Stu1 and by transforming, with the plasmid thus linearized, a yeast strain carrying a point mutation in the trp1 gene.

6.1.1. Construction of the Plasmid pXylex:

The DNA fragment containing the promoter of the GAL1 gene whose transcription activating sequences have been replaced by E. coli LexA protein binding sites, whose sequence is that deposited at the EMBL bank, under the identifier “ECLEXA1”, under the No. J01643, was amplified by PCR from the plasmid pSH18-34 (Hanes S. D. et al., (1989), Cell, 57, 1275-1293) using the oligonucleotides “olGAL1” having the sequence SEQ ID No. 12 5′GCCAAGCTTCTCCTTGACGTTAAAGTA3′ and “olGAL10” having the sequence SEQ ID No. 13 5′GCCGGATCCTTTGTAACTGAGCTGTCA3′. The amplified DNA was digested with the restriction enzymes BamHI and HindIII and inserted into the vector pEMBLYe31-X (Jacquemin-Faure I. et al., (1994), Mol. Gen. Genet., 244, 519-529) digested beforehand with the enzymes BamHI and HindIII, producing the vector pXylex.

6.1.2. Construction of the Plasmid pYi39:

The vector pFL39 was digested with the enzyme ClaI and was self-ligated and a plasmid having lost the ClaI-ClaI fragment containing the original ars-cen replication origin was selected, producing the vector pYi39.

6.1.3. Construction of the Plasmid pYi39Xylex:

The BamHI-PstI DNA fragment of the vector pXylex prepared beforehand and containing the XylE gene whose sequence is that deposited at the EMEL bank, under the identifier “PPXYLE”, under the No. V01161 preceded by the GAL1-LexA promoter was isolated and inserted into the plasmid pYi39 digested beforehand with the enzymes BamHI and PstI, producing the plasmid pYi39Xyl-Lex.

6.2. Construction of the Plasmid pYi44Xylex:

6.2.1. Construction of the Plasmid pYi44:

The vector pFL44 was digested with the enzyme ClaI and was self-ligated and a plasmid having lost the ClaI-ClaI fragment containing the original 2 μm replication origin was selected, producing the vector pYi44.

6.2.2. Construction of the Plasmid pYi44Xylex

The BamHI-PstI DNA fragment of the vector pXylex prepared according to the procedure described at point 6.1.1 and containing the XylE gene preceded by the GAL1-LexA promoter region was isolated and inserted into the plasmid pYi44 digested beforehand with the enzymes BamHI and PstI, producing the plasmid pYi44Xyl-Lex.

6.3. Construction of the Plasmid pLexM4-7:

6.3.1. Use:

It allows the expression in the yeast S. cerevisiae of the Met4 protein containing at its amino-terminal end the 202 residues of the E. coli LexA protein. The hybrid protein thus synthesized is capable of binding to DNA regions comprising the LexA operators.

6.3.2. Construction:

The EcoRI-BamHI DNA fragment of the vector pLexM4-1 encoding amino acids 15 to 666 of the Met4 protein was cloned into the plasmid pBTM116 (Margottin M. et al., (1998), Molecular Cell, 1, 565-574) digested beforehand with the enzymes EcoRI and BamHI, producing the plasmid pLexM4-7 This plasmid can replicate autonomously in yeast.

6.4. Construction of the Plasmid pLexM4Δ239

6.4.1. Use:

It allows the expression in the yeast Saccharomyces cerevisiae of a derivative of the Met4 protein from which there have been amputated its residues 212 to 231 comprising at its amino terminal end the 202 residues of the E. coli LexA protein. The hybrid protein thus synthesized is capable of binding to DNA regions comprising LexA operators.

6.4.2. Construction:

The EcoRI-BmHI DNA fragment of the vector pLexM4Δ30 encoding a derivative comprising amino acids 15 to 211 and 232 to 666 of the Met4 protein was cloned into the plasmid pBTM116 (Margottin M. et al., (1998), reference cited) digested beforehand with the enzymes EcoRI and BamHI, producing the plasmid pLexM4Δ239. This plasmid can replicate autonomously in yeast.

6.5. Construction of the Plasmid pM16Z1:

The DNA fragment containing the promoter of the MET16 gene (comprising nucleotides −535 to +3, numbered from the initiation codon of the MET16 gene) was amplified by PCR from the plasmid pM16-1 (Hanes S. D. et al., (1989), reference cited) using the oligonucleotides “M16OL2” having the sequence SEQ ID No. 14 5′CAACGAAGGATCCAATAATCGAAGCC3′ and “M16OL4” having the sequence SEQ ID No. 15 5′GGGGAATTCCTTCATTTTATGAGTTGCT3′. The amplified DNA was digested with the restriction enzymes BamHI and EcoRI and inserted into the vector Yep356R (Myers A. M. et al. (1986), Gene, 45, 299-310) digested beforehand with the enzymes BamHI and EcoRII, producing the vector pM16Z1. This plasmid can replicate autonomously in yeast and allows the expression of E. coli β-galactosidase under the control of the MET16 promoter.

6.6. Construction of the Plasmid pM16Xyl1:

The DNA fragment containing the promoter of the MET16 gene (comprising nucleotides −535 to −1, numbered from the initiation codon of the MET16 gene) was amplified by PCR from the plasmid pM16-1 (Thomas D. et al. (1990), J. Biol. Chem., 265, 15518-15524) using the oligonucleotides “M16OL5” having the sequence SEQ ID No. 16 5′CAACGAAGCTTTCAATAATCGAAGCACTTGG3′ and “M16OL6” having the sequence SEQ ID No. 17 5′TTTATGAGAAGCTTTGGGTTGATACCTTTGC3′. The amplified DNA was digested with the restriction enzyme HindIII and inserted into the vector pUC9-LEU2-X (Jacquemin-Faure I. et al., (1994), reference cited) producing the plasmid pM16Xyl1. The integration of this plasmid into the yeast genome at the LEU2 locus can be directed by linearizing this plasmid with the enzyme Asp718 and by transforming, with the plasmid thus linearized, a yeast strain carrying a point mutation in the leu2 gene. Thus, this plasmid makes it possible to obtain a modified yeast strain stably expressing P. putida catechol oxidase placed under the control of the MET16 gene.

### EXAMPLE 7

### Yeast Strains Expressing a GFP-Met4 Hybrid Protein

These strains carry at the level of the URA3 locus (chromosome V, left arm) an artificial gene allowing the expression of a Met4 protein, modified or otherwise, under the control of the promoter of the GAL1 gene.

The correct integration into the URA3 locus of the GAL-Met4-GFP fusions was verified by conventional molecular biology and genetic techniques.

The strains which were prepared are assembled in table I below.

TABLE I

| Name of the | | Characteristics of the |
|---|---|---|
| yeast strain | Plasmid used | strain (genotype) |
| C300 | pGal306GFPMet4 | Mata, his3, leu2, trp1, |
| | | ura3::galI-GFP- |
| | | Met4::URA3 |
| C301 | pGal306GFPMet4 | Matα, his3, leu2, trp1, |
| | | met4::TRP1, ura3::galI- |
| | | GFP-Met4::URA3 |
| C302 | pGal306GFPMet4 | Matα, his3, leu2, trp1, |
| | | met30-2, ura3::gal1- |
| | | GFP-Met4::URA3 |
| C304 | pGal306GFPMet4Δ12 | Matα, his3, leu2, trp1, |
| | | met4::TRP1, ura3::gal1- |
| | | GFP-Met4Δ12::URA3 |
| C305 | pGal306GFPMet4Δ30 | Matα, his3, leu2, trp1, |
| | | met4::TRP1, ura3::gal1- |
| | | GFP-Met4Δ30::URA3 |
| C307 | pGal306GFPMet4Δ37 | Matα, his3, leu2, trp1, |
| | | met4::TRP1, ura3::gal1- |
| | | GFP-Met4Δ37::URA3 |
| C312 | pGal306GFPMet4 | Mata, his3, leu2, trp1, |
| | | ura3::gal1-GFP- |
| | | Met4::URA3 |
| C319 | pGal306GFPMet4 | Mata, his3, leu2, trp1, |
| | | cdc34-2, ura3::gal1- |
| | | GFP-Met4::URA3 |
| C323 | pGal306GFPMet4 | Matα, his3, leu2, trp1, |
| | | cdc53-1, ura3::gal1- |
| | | GFP-Met4::URA3 |
| C327 | pGal306GFPMet4 | Matα, his3, leu2, trp1, |
| | | skp1-11, ura3::gal1- |
| | | GFP-Met4::URA3 |

7.1. Preparation of the Strains from pGal306GFPMet4:

The integration of this plasmid into the yeast genome at the URA3 locus can be directed by linearizing this plasmid with the enzyme Stu1 and by transforming, using the plasmid thus linearized, a yeast strain carrying a point mutation in the ura3 gene. The GFP-Met4 fusion thus integrated into the URA3 chromosomic locus is expressed under the control of the GAL1 promoter.

7.2. Preparation of the Strains from pGal306GFPMet4Δ12:

The strains are transformed according to the procedure described at point 7.1. from the plasmid pGal306GFPMet4Δ12.

7.3. Preparation of the Strains from pGal306GFPMet4Δ30:

The strains are transformed according to the procedure described at point 7.1. from the plasmid pGal306GFPMet4Δ30.

7.4. Preparation of the Strains from pGal306GFPMet4Δ37:

The strains are transformed according to the procedure described at point 7.1. from the plasmid pGal306GFPMet4Δ37.

### EXAMPLE 8

### Yeast Strains Carrying the XyLE Gene Encoding Catechol Oxidase Under the Control of LexA Operators

8.1. Preparation of the Strains:

These strains carry at the level of the URA3 locus (chromosome V, left arm) or at the level of the TRP1 locus (chromosome IV, right arm) an artificial gene comprising the Pseudomonas putida XylE gene placed under the control of LexA operators. The expression of this gene is directed by the LexA-Met4 hybrid protein expressed from the replicative plasmid pLexM4-7.

The correct integration into the URA3 locus of the GAL-Met4-GFP fusions was checked by conventional molecular biology and genetic techniques.

The strains which were prepared are assembled in table II below.

TABLE II

| Name of the | Plasmid | Characteristics of the strain |
|---|---|---|
| yeast strain | used | (genotype) |
| C190 | pYi44Xylex | Mata, ade2, his3, leu2, trp1, |
| | | ura3::LeAₒₚ-XylE::URA3 |
| C192 | PYi39Xylex | Mata, ade2, his3, leu2, trp1, |
| | | Trp1::LeXAₒₚ-XylE::TRP1 |
| C193 | pYi44Xylex | Mata, ade2, his3, leu2, trp1, |
| | | met4::TRP1, ura3::LeAₒₚ- |
| | | XylE::URA3 |

8.2. Preparation of the Strains from the Plasmid pYi44XyLex:

The integration of this plasmid into the yeast genome at the URA3 locus can be directed by linearizing this plasmid with the Stu1 enzyme and by transforming, with the plasmid thus linearized, a yeast strain carrying a point mutation in the ura3 gene (Orr-Weaver T. L. et al. (1983) and Rothstein R. (1991), references cited).

8.3. Preparation of the Strains from the Plasmid pYi39XyLex

The integration of this plasmid into the yeast genome at the TRP1 locus can be directed by linearizing this plasmid with the Stu1 enzyme and by transforming, with the plasmid thus linearized, a yeast strain carrying a point mutation in the trp1 gene (Orr-Weaver T. L. et al. (1983) and Rothstein R. (1991), references cited).

### EXAMPLE 9

### Yeast Strain Carrying the XylE Gene Encoding Catechol Oxidase Under the Control of the Promoter of the MET25 Gene

9.1. Preparation of the CC634-2D Strain:

This strain is derived from the cross between the CI2-11D strain (Jacquemin-Faure I. et al., (1994), reference cited) and the CD106 strain (Thomas D. et al., (1992), reference cited). It contains, integrated at the LEU2 locus (chromosome III, left arm), an XylE gene placed under the control of the promoter of the MET25 gene.

9.2. Characteristics of the CC634-2D Strain:

The genotype of this strain is the following: Mata, ade2, his3, trp1, ura3, met4::TRP1, leu2::proMet25-XylE::LEU2.

### EXAMPLE 10

### Yeast Strain Carrying the HIS3 Gene Under the Control of LexA Operators

These strains carry at the level of the LYS2 locus (chromosome II, right arm) an artificial gene comprising the S. cerevisiae HIS3 gene placed under the control of LexA operators. The expression of this gene is directed by the LexA-Met4 hybrid protein expressed from the replicative plasmid pLexM4-7.

10.1. Preparation of the Strains

10.1.1. Preparation of the CC817-4A Strain:

This strain is derived from the cross between the L40 strain (Holenberg S. M. et al., (1995), Mol. Cell Biol., 15, 3813-3822) and the CC816-1D strain having the genotype Matα, ade2, leu2, lys2, trp1, ura3.

10.1.2. Preparation of the CY25-1D Strain:

This strain is derived from the cross between the CC817-4A strain prepared above and the CD106 strain (Thomas D. et al., (1992), reference cited)

10.2. Characteristics of the Strains:

10.2.1. Characteristics of the CC817-4A Strain:

The genotype of this strain is the following: Mata, ade2, his3, leu2, trp1, ura3, lys2::LexAop-HIS3::LYS2

10.2.2. Characteristics of the CY25-1D Strain:

The genotype of this strain is the following: Mata, ade2, his3, leu2, trp1, ura3, met32::URA3, lys2::LexAop-His3::LYS2

### EXAMPLE 11

### Yeast Strain Carrying the LacZ Gene Under the Control of LexA Operators

These strains carry at the level of the URA3 locus (chromosome V, left arm) an artificial gene comprising the E. coli LacZ gene placed under the control of LexA operators. The expression of this gene is directed by the LexA-Met4 hybrid protein expressed from the replicative plasmid pLexM4-7.

11.1. Preparation of the CC801-3B Strain

This strain is derived from the cross between the L40 strain (Hollenberg S. M. et al., (1995), reference cited) and the CD106 strain (Thomas D. et al., (1992), reference cited).

11.2 Characteristics of the CC801-3B Strain:

The genotype of this strain is the following: Mata, ade2, his3, leu2, trp1, ura3 met4::TRP1, ura3::LexAop-LacZ::URA3

### EXAMPLE 12

### Determination of the Level of Expression and of the Stability of the Proteins Expressed from the Hybrid Sequences Contained in the Plasmids According to the Invention

12.1. Procedure:

12.1.1. Determination of the Stability of the Hybrid Proteins:

a) Visualization with the Hemagglutinin (Ha) Antigenic Marker:

Yeast cells containing a plasmid encoding the proteins labeled with Ha, under the control of the GAL1 promoter, are cultured in a medium containing raffinose.

The induction of the hybrid protein, expressed under the control of the GAL1 promoter, is carried out for 2 to 10 hours, by transferring the cells into a medium containing 2 to 5% galactose.

Samples were collected at 0, 5, 10, 20, 40, 60 and 80 minutes after the addition of glucose and optionally methionine, at a repressive concentration of between 0.05 mM and 25 mM.

The stability of the hybrid proteins is measured by reaction with anti-Ha antibodies, according to conventional techniques.

b) Visualization with the GFP Protein:

Yeast cells containing a plasmid encoding the proteins labeled with the GFP protein, under the control of the GAL1 promoter, are cultured in a medium containing raffinose and the induction of the hybrid protein is carried out according to the procedure described above.

The measurement of fluorescence is carried out after 20 minutes of incubation in the presence or in the absence of repressive concentrations of methionine.

c) Visualization with Catechol Oxidase:

The activity of the reporter gene LexAopXyle is measured in a yeast strain (C190) expressing the hybrid protein encoded by the plasmid pLexMet4-7 and cultured under the conditions described above.

The activity is measured by a visualization technique based on measuring catechol oxidase according to conventional techniques.

12.1.2. Measurement of the Total RNAs:

It is carried out by conventional techniques known to a person skilled in the art.

12.2. Results:

12.2.1. Stability of the Hybrid Proteins:

a) Visualization by the Hemagglutinin (Ha) Antigenic Marker

They are assembled in FIGS. 1 and 3B.

Expression of the Ha-Met4 hybrid protein

The Ha-Met4 protein has a half-life of the order of 20 minutes in the absence of methionine (FIG. 1A, −Met) and a half-life of the order of 5 minutes under repressive conditions, that is to say in the presence of methionine (FIG. 1A, +Met).

The degradation of the Ha-Met4 protein is almost complete under repressive conditions after 20 minutes (FIG. 1A, +Met)

Expression of the Ha-Met28 hybrid protein

The Ha-Met28 protein has a half-life of the order of 20 minutes in the absence of methionine (FIG. 1A, −Met).

Repressive conditions do not modify the half-life of the Ha-Met28 protein (FIG. 1A, +Met).

Expression of the Ha-Met30 and Ha-Met30ΔF hybrid proteins:

The Ha-Met30 protein has a half-life of the order of 20 minutes in the absence of methionine (FIG. 3B, −Met).

Repressive conditions do not modify the half-life of the Ha-Met30 hybrid protein (FIG. 3B, +Met).

The Ha-Met30ΔF hybrid protein appears less stable than the Ha-Met30 hybrid protein, but repressive conditions do not reduce the stability of this hybrid protein.

b) Visualization with Hybrid Proteins Comprising GFP

The results are illustrated in FIGS. 2 and 3B.

Location of the GFP-Met4 hybrid protein

The GFP-Met4 hybrid protein is located in the nucleus when the cells are cultured in the absence of methionine.

On the other hand, under repressive conditions, that is to say in the presence of methionine, this location is no longer observed (FIG. 2A).

These results are in agreement with a rapid degradation of the hybrid protein under repressive conditions.

Location of the GFP-Met28 hybrid protein

The GFP-Met28 hybrid protein is always present in the nucleus, whether in the absence or in the presence of methionine (FIG. 2B).

Location of the GFP-Met30 hybrid protein

The GFP-Met30 hybrid protein is always present in the nucleus, whether in the absence or in the presence of methionine (FIG. 3A).

12.2.2. Expression of Total RNAs

The results are illustrated in FIGS. 1B and 1C

The levels of Met4 and of Met28 allow the activation of the transcription of the target genes.

Neither the expression of MET4, nor that of MET28, under the control of the GAL1 promoter, modifies the repression induced by the addition of methionine.

12.2.3. Visualization with Catechol Oxidase

The results are illustrated in FIG. 4.

In the absence of methionine, the cells are stained yellow, proof that the protein is expressed.

In the presence of methionine, the cells are white because of the repression.

1. A method for cell screening of compounds capable of acting on pathologies linked to the dysfunction of the ubiquitin-proteasome cascade in humans, said method comprising,
(i) bringing the product to be tested into contact with a modified yeast strain containing (a) a hybrid sequence comprising a sequence encoding a Met4 protein, in its wild-type form or in a mutated form involved in the transcriptional activation of the MET genes, fused in phase with at least one sequence encoding an appropriate marker, said hybrid sequence being expressed under the control of a promoter active in yeast and optionally (b) a reporter transcriptional system consisting of a reporter gene placed under the control of an appropriate operator sequence or of an appropriate yeast promoter,
(ii) adding methionine at repressive concentrations of between 0.03 mM and 20 mM, or at nonrepressive concentrations, and
(iii) determining the level of expression and stability of the protein expressed from the hybrid sequence, either by visualization and/or quantification, or by determination of the activity of the reporter gene.

2. The method as claimed in claim 1, comprising, in parallel,
(iv) bringing the product to be tested into contact with a modified yeast strain containing a hybrid sequence comprising a sequence encoding a Met30 protein, in its wild-type form or in a mutated form involved in the biosynthesis of sulphur amino acids, fused in phase with at least one sequence encoding an appropriate marker, said hybrid sequence being expressed under the control of a promoter active in yeast,
(v) adding methionine at repressive concentrations of between 0.03 mM and 20 mM, or at nonrepressive concentrations, and
(vi) determining the level of expression and stability of the protein expressed from the hybrid sequence, either by visualization and/or quantification, or by determination of the activity of the reporter gene.

3. The method as claimed in claim 1, further comprising an additional means using a cellular system of control, said means comprising,
(vii) bringing the product to be tested into contact with a modified yeast strain containing a hybrid sequence comprising a sequence encoding a Met28 protein, in its wild-type form or in a mutated form involved in the transcriptional activation of the MET genes, fused in phase with at least one sequence encoding an appropriate marker, said hybrid sequence being expressed under the control of a promoter active in yeast,
(viii) adding methionine at repressive concentrations of between 0.03 mM and 20 mM, or at nonrepressive concentrations, and
(ix) determining the level of expression and stability of the protein expressed from the hybrid sequence, either by visualization and/or quantification, or by determination of the activity of the reporter gene.

4. The method as claimed in claim 1, further comprising an additional means using an acellular system of control which is based on measuring the levels of transcription of the hybrid sequences and of the metabolic genes MET16 and MET25, said means comprising
(x) extracting the total RNAs of the modified strains used either in (i), or in (iv), or in (vii) and
(xi) measuring the levels of transcription of the hybrid sequence and that of the metabolic genes MET16 and MET25.

5. The method as claimed in claim 1, wherein the marker used for the construction of the hybrid sequence is chosen from the group consisting of: the antigenic peptides, the intrinsic fluorescence proteins, the proteins with measurable enzymatic activity and the DNA-binding factors.

6. The method as claimed in claim 1, wherein the promoter allowing the expression of the hybrid protein is chosen from the group consisting of inducible promoters active in S. cerevisiae and constitutive promoters.

7. The method as claimed in claim 1, wherein the reporter gene present in the transcriptional reporter system is chosen from the group consisting of the reporter genes whose activity can be visualized by a calorimetric method, and the metabolic genes, whose activity can be measured by a growth test.

8. The method as claimed in claim 1, wherein said reporter gene is placed under the control, either of the promoter of a MET gene, or of LexA operators.

9. The method as claimed in claim 8, wherein said MET gene is MET3, MET10, MET16, MET25 or MET28.

10. A method for cell screening of compounds capable of acting on the pathologies linked to the dysfunction of the ubiquitin-proteasome cascade in humans, said method comprising
(xii) bringing the product to be tested into contact with a modified yeast strain containing a reporter transcriptional system consisting of a reporter gene placed under the control of a promoter selected from the group consisting of inducible promoters active in S. cerevisiae and constitutive promoters.
(xiii) adding methionine at repressive concentrations of between 0.03 mM and 20 mM, or at nonrepressive concentrations, and
(xiv) comparing the activity of the reporter gene in repressive conditions to the activity of the reporter gene in nonrepressive condition.

11. The method as claimed in claim 10, wherein the promoter is activated by the Met4 transcription factor.

12. The method as claimed in claim 10, wherein the reporter gene present in the transcriptional reporter system is chosen from the group consisting of the reporter genes whose activity can be visualized by a calorimetric method, and the metabolic genes, whose activity can be measured by a growth test.

13. The method as claimed in claim 10, wherein said reporter gene is placed under the control, either of the promoter of a MET gene, or of LexA operators.

14. The method as claimed in claim 13, wherein said MET gene is MET3, MET10, MET16, MET25 or MET28.

15. A plasmid, comprising a hybrid sequence comprising a sequence encoding a Met4 protein in its wild-type form or in a mutated form involved in the transcriptional activation of the MET genes, fused in phase with at least one sequence encoding an intrinsic fluorescence protein, it being possible for said hybrid sequence to be expressed in yeast under the control of a constitutive or inducible promoter.

16. The plasmid as claimed in claim 15, wherein said intrinsic fluorescence protein is the GFP protein and said promoter is a constitutive promoter selected from MET4, MET28 and MET30.

17. The plasmid as claimed in claim 15, wherein said intrinsic fluorescence protein is the GFP protein and said promoter is the GAL1 inducible promoter.

18. A yeast strain stably modified with at least one plasmid as claimed in claim 15.

19. A plasmid, comprising a hybrid sequence comprising a sequence encoding a Met28 protein in its wild-type form or in a mutated form involved in the transcriptional activation of the MET genes, fused in phase with at least one sequence encoding an intrinsic fluorescence protein, it being possible for said hybrid sequence to be expressed in yeast under the control of a constitutive or inducible promoter.

20. The plasmid as claimed in claim 19, wherein said intrinsic fluorescence protein is the GFP protein and said promoter is a constitutive promoter selected from MET4, MET28 and MET30.

21. The plasmid as claimed in claim 19, wherein said intrinsic fluorescence protein is the GFP protein and said promoter is the GAL1 inducible promoter.

22. A yeast strain stably modified with at least one plasmid as claimed in claim 19.

23. A plasmid comprising a hybrid sequence comprising a sequence encoding a Met30 protein in its wild-type form or in a mutated form involved in the biosynthesis of sulphur amino acids, fused in phase with at least one sequence encoding an intrinsic fluorescence protein, it being possible for said hybrid sequence to be expressed in yeast under the control of a constitutive or inducible promoter.

24. The plasmid as claimed in claim 23, wherein said intrinsic fluorescence protein is the GFP protein fused with a peptide comprising 3 hemagglutinin antigenic units and said promoter is the GAL1 inducible promoter.

25. The plasmid as claimed in claim 23, wherein said intrinsic fluorescence protein is the GFP protein and said promoter is a constitutive promoter selected from MET4, MET28 and MET30.

26. The plasmid as claimed in claim 23, wherein said intrinsic fluorescence protein is the GFP protein and said promoter is the GAL1 inducible promoter.

27. A yeast strain stably modified with at least one plasmid as claimed in claim 23.
